(19) United States
(12) Patent Application Publication
(10) Pub. No.: US 2023/0301931 A1
(43) Pub. Date: Sep. 28, 2023

(54) NANOPARTICULATE SYSTEM FOR TREATING ORAL CANCER

(71) Applicant: THE TRUSTEES OF COLUMBIA UNIVERSITY IN THE CITY OF NEW YORK, New York, NY (US)

(72) Inventors: Kam W. LEONG, New York, NY (US); Divya BHANSALI, New York, NY (US); Tianyu LI, New York, NY (US)

(21) Appl. No.: 18/041,618

(22) PCT Filed: Aug. 16, 2021
(86) PCT No.: PCT/US2021/046153

§ 371 Date: Feb. 14, 2023

Related U.S. Application Data
(60) Provisional application No. 63/079,528, filed on Sep. 17, 2020.
(60) Provisional application No. 63/065,806, filed on Aug. 14, 2020.

(51) Int. Cl. A61K 9/51; A61K 45/06

(52) U.S. Cl. CPC A61K 9/5146; A61K 9/5123; A61K 45/06; B82Y 5/00

(57) ABSTRACT

The nanoparticulate system for delivering treating agents may be used, for example, for treating inflammation, or for treating cancer, such as oral cancer, and associated inflammation The system used in this manner provides a solution to oral cancer and its associated metastasis by providing a targeted therapy to deliver chemotherapeutics. The nanoparticulate system is in the form of cholesterol-modified polyamidoamine-G3 nanoparticles (PAMAM-Chol NPs), which are used as a carrier for at least one drug. The at least one drug may include at least one cancer drug, at least one corticosteroid, at least one anabolic steroid, at least one hormone (natural or synthetic), and combinations thereof.

### CROSS-REFERENCE TO RELATED APPLICATION

This application claims the benefit of U.S. Provisional Patent Application No. 63/079,528, filed on Sep. 17, 2020, and U.S. Provisional Patent Application No. 63/065,806, filed on Aug. 14, 2020.

### GOVERNMENT FUNDED RESEARCH

This invention was made with government support under NS102722, DE026806, and DK118971 awarded by National Institutes of Health, and W81XWH-18-1-0431 awarded by ARMY/MRMC. The government has certain rights in the invention.

### TECHNICAL FIELD

The disclosure of the present patent application relates to cholesterol-modified polyamidoamine (PAMAM)-G3 nanoparticles (PAMAM-Chol NPs, or PAMAM-CHOL particles) for use as a carrier of drugs, such as without limitation chemotherapy and anti-nociceptive drugs, cancer treatments and treatments of cancer associated pain, as well as corticosteroids, anabolic steroids, and hormones. These PAMAM-CHOL particles also may be used as a scavenger for treating oral cancer, inflammation and associated pain.

As used herein, “polyamidoamine-G3” refers to the third generation polyamidoamine dendrimer. The disclosure of the present patent application further relates to the use of nucleic acid-binding polymers (NABP) and nanoparticles (NABPN) as anti-inflammatory agents to scavenge damage-associated molecular patterns (DAMPs) and deliver pain receptor antagonists and chemotherapeutics at the same time, particularly as a therapeutic strategy to manage primary and metastatic tumor progression as well as to treat or address associated inflammation or pain. These nanoparticles may also be used to administer various combinations of drugs, including without limitation chemotherapy drugs, corticosteroids, steroids, and hormones.

### BACKGROUND ART

Various forms and structures of nanoparticles have been prepared for treating different conditions, or for delivery of various treating agents. One such treatment uses a delivery system that focuses, for example, on lipid nanoparticle compositions that include albumin, for antisense oligonucleotides delivery. The subject matter requires a cationic liposome, a targeting agent, and a net positively-charged core comprising an albumin-polycation conjugate. The polycation may be poly(amido amine) (PAMAM) dendrimers. Another system focuses on methods for treating acute myeloid leukemia using nanoparticle complexes including PAMAM dendrimers complexed with microRNA-22(miR-22). Another system focuses on targeting glioblastoma stem cells through the TLX-TET3 axis, providing, for example, treatment of brain cancer using a specific shRNA or siRNA that modulates TLX activity. The treatment may be complexed with a nanoparticle such as PAMAM dendrimer.

U.S. Pat. No. 9,168,225 focuses on nano-hybrid delivery systems for sequential utilization of passive and active targeting. The delivery system uses, for example, a multivalent polymeric scaffold nanocore consisting of branched polyethyleneimine or PAMAM dendrimer with a therapeutic agent and a targeting agent covalently attached thereto, and also requires an outer shell encapsulating the polymeric scaffold nanocore, the therapeutic agent and the targeting agent, wherein the shell consists of poly-(lactic acid-co-glycolic acid), polyethylene glycol-b-polylactide-co-glycolide, polyethylene glycol-b-poly-L-lactic acid, or a unilamellar liposome consisting of 1,2-distearoyl-sn-glycero-3-phosphoethanolamine-N-mPEG-2000, 1,2-dioleoyl-sn-glycero-3-phosphocholine, 1,2-dioleoyl-sn-glycero-3-phospho-(1′-rac-glycerol) and cholesterol.

U.S. Patent Application Publication No. 2020/0069594 focuses on hybrid exosomal-polymeric (HEXPO) nano-particles for delivery of RNAi therapeutics. The lipid-based nanoparticles may include a core comprising a cationic polymer and a therapeutic agent, and a lipid coating comprising an exosomes-derived membrane. The cationic polymer may be PAMAM.

Nano-particle treating and delivery systems may be used in a wide variety of treatments, and, when used to deliver an independent treating agent, a wide variety of treating agents. Certain conditions are particularly appropriate for using nano-particle drug delivery systems.

Cancer is typically treated using traditional chemotherapy drugs, which carry numerous systemic side-effects, including inflammation and pain. The pain associated with cancer, and with the chemotherapy treatment, is often treated using non-steroidal anti-inflammatories (NSAIDs), such as ibuprofen, aspirin, etc., opioids, such as morphine, codeine, oxycodone, etc., antiepileptics (e.g., Gabapentin), steroids (e.g., prednisone, dexamethasone, etc.), and other typical pain and side-effect amelioration drugs. Such treatments, particularly with regard to the pain treatments, typically require high dosages to be used and carry a high risk of addiction, as well has dangerous side-effects. The ability to target delivery of the treating agent helps avoid the negative results often associated with high systemic or regionalized concentrations otherwise often associated with methods for delivering sufficient concentrations of the treating agent.

Oral cancer is one of the most common forms of cancer in both men and women. Over 40% of oral cancer patients eventually develop metastatic disease and die. Surgery, in conjunction with chemotherapy and radiation, can remove the primary tumor but creates a lot of pain and inflammation and can increase the likelihood of metastasis. Tumor cells, as well as those in the tumor microenvironment, release their contents into the body when killed through chemotherapy and radiation. These contents include damage-associated molecular patterns (DAMPs) in the form of fragmented nucleic acids and associated proteins that may stimulate the immune system to promote inflammation and pain.

Thus, a nanoparticulate system for delivering treating agents for treating conditions such as inflammation or pain, or treating cancer, and specifically, for example, oral cancer, and associated inflammation and pain, while solving the aforementioned problems is desired.

### DISCLOSURE

The nanoparticulate system provides a targeted therapy to treat pain, and to deliver anti-nociceptive drugs (non-opiates), and chemotherapeutics, or combinations thereof. The treatment is in the form of cholesterol-modified polyamidoamine-G3 nanoparticles (PAMAM-Chol NPs), which are used as a carrier for at least one drug. As used herein, “polyamidoamine-G3” refers to the third generation polyamidoamine dendrimer.

When used for treating oral cancer and its associated inflammation and metastasis, the nanoparticulate system provides a solution by providing at least one drug that may be, for example, a chemotherapy drug, a protease-activated receptor 2 antagonist (e.g., I-343 or I-560), or a combination thereof. The at least one drug may also include at least one cancer drug, at least one corticosteroid, at least one anabolic steroid, at least one hormone (natural or synthetic), and combinations thereof. It should be understood that the term “cancer drug”, as used herein, is a therapeutic agent for treating cancer, including such agents described as chemotherapy drugs, anti-cancer drugs, anti-tumor drugs, and antineoplastic drugs.

Non-limiting examples of the at least one cancer drug include coxorubicin, paclitaxel, camptothecin, docetaxel, pemetrexed, curcumin, gemcitabine, dabrafenib, dexamethasone, gefitinib, lenvatinib, methotrexate, thalidomide, vinblastine, vincristine, cyclophosphamide, ifosfamide, glyciphosphoramide, nimustine, carmustine, comustine, 5-fluorouracil, doxifluridine, mercaptopurine, cisplatin, and combinations thereof.

Non-limiting examples of the at least one corticosteroid include cortisone, prednisone, prednisolone, methylprednisolone, dexamethasone, betamethasone, hydrocortisone, and combinations thereof. Non-limiting examples of the at least one anabolic steroid include anadrol, oxandrin, dianabol, winstrol, deca-durabolin, equipoise, and combinations thereof.

Non-limiting examples of the at least one hormone include alclometasone, prednisone, dexamethasone, triamcinolone, cortisone, fludrocortisone, dihydrotachysterol, oxandrolone, oxabolone, testosterone, nandrolone, diethylstilbestrol, ethinyl estradiol, norethisterone, medroxyprogesterone acetate, hydroxyprogesterone caproate, estrogen, estradiol, estriol, estrone, cortisol, 11-deoxycortisol, aldosterone, corticosterone, 11-deoxycorti-costerone, aldosterone, progestin, pregnenolone, progesterone, 17α-hydroxy progesterone, 17α-hydroxy pregnenolone, dehydroepiandrosterone, androstenedoil, androstenedione, dihydrotestosterone, melatonin, thyroxine, and combinations thereof.

The nanoparticulate system may also be loaded with at least one drug intended specifically for pain relief. Non-limiting examples include NSAIDS (nonlimiting examples include Indomethacin, Sulindac, Etodolac, Tolmetin, Ketorolac, Oxaprozin, Fenoprofen, Flurbiprofen, Ibuprofen, Ketoprofen, Naproxen, Nambumetone, Meclofenamate, Diclofenac, Piroxicam, Meloxicam, Celecoxib, Rofecoxib, Valdecoxib, Aspirin, and combinations thereof), opioids (nonlimiting examples include Fentanyl, Alfentanil, Sufentanil, Remifentanil, Methadone, and combinations thereof), and local anesthetics (nonlimiting examples include Dibucaine, Bupivacaine, Lidocaine, Procaine, Mepivacaine, Rapivacaine, and combinations thereof). Of course, suitable steroids, including without limitation the corticosteroids and anabolic steroids listed above and the steroids listed below, may also be useful for indirectly providing pain relief.

As a non-limiting example of dual-drug treatment, the PAMAM-Chol NPs may be loaded with a cancer drug and a steroid. For example, steroids, such as dexamethasone, prednisolone, methylprednisolone, and/or hydrocortisone, are often given to patients to help reduce side effects of chemotherapy drugs, as well as reduce inflammation and help with appetite and energy levels. Thus, as a non-limiting example, both a cancer drug (i.e., a chemotherapy drug) and a steroid can both be loaded on the PAMAM-Chol NPs. As a further alternative example, steroids or hormones could be loaded with one or more cancer drugs, and this combination could be loaded on the PAMAM-Chol NPs. It should, however, be understood that any suitable type of therapeutic agent or treatment may be loaded on the PAMAM-Chol NPs, and that the choice of drugs is not limited to the examples described above.

The nanoparticulate system when used for treating oral cancer and associated inflammation and/or pain is a targeted therapy in a nano-delivery platform that is able to deliver chemotherapy and pain relief to target cells, thus ensuring concentrated therapeutic delivery and non-opioid pain relief. By targeting cancer cells and pain receptors, the delivery system provides a high local concentration of the anti-nociceptive or chemotherapeutic cargo where it is most effective, while helping maintain low systemic concentrations of the treating agents. This may allow for lower dosages and decreased off-target (i.e., addiction) effects, and provide alternative tools to treat oral cancer and its associated inflammation or pain. It should be understood that the treatment is not limited to oral cancers and may be used, for example, to treat pancreatic cancer, lung cancer, other painful cancers, general inflammatory diseases, and as a combined delivery system for pain treatment and therapeutic agents for diseases that cause chronic pain.

These and other features of the present subject matter will become readily apparent upon further review of the following specification.

### BRIEF DESCRIPTION OF THE DRAWINGS

FIG. 1A illustrates the structure and formulation of polyamidoamine (PAMAM)-Chol(5) Polymer.

FIG. 1B is a set of transmission electron microscope (TEM) images of PAMAM-Chol NPs.

FIG. 1C is a graph illustrating the DNA binding efficiency of PAMAM-Chol NPs. This is directly related to their ability to bind nucleic acid DAMPs.

FIG. 1D is a graph illustrating the results of a Cck8 cytotoxicity assay of PAMAM-Chol NPs.

FIG. 2A is a graph showing cfDNA levels of human samples from patients with oral tongue squamous cell carcinoma (OTSCC).

FIG. 2B is a graph showing activation of TLR 9 by OTSCC supernatants. FIG. 2C is a graph showing results of inhibition of TLR 9 activation by PAMAM-Chol NPs.

FIG. 2D shows a set of wound-healing assay images, with the assay performed using confluent serum-starved HSC-3 cells either untreated or treated with 1 μg/mL cfDNA and 20 or 50 μg/mL dendrimers, illustrating that PAMAM-Chol NPs are able to successfully mediate cell migration.

FIG. 2E is a graph showing a quantification of the wound healing assay of FIG. 2D, showing PAMAM-Chol NPs are successfully able to mediate damage associated molecular pattern (DAMP) induced cell migration, where the quantification is shown in the form of wound width relative to a control group, and produced using Image J software.

FIG. 3 diagrammatically illustrates the use of scavengers to inhibit TLR activation and inflammation.

FIG. 4A is a plot showing nucleic acid (NA) levels in plasma and saliva of patients with oral squamous cell carcinoma, where healthy or tumor tissues (blood and saliva) were incubated in culture media for 24 hours before the supernatant was collected and the cfRNA was measured by Quanti-iT RNA (Thermo).

FIG. 4B is a plot showing nucleic acid (NA) levels in plasma and saliva of patients with oral squamous cell carcinoma, where healthy or tumor tissues (blood and saliva) were incubated in culture media for 24 hours before the supernatant was collected and the miRNA was measured by a miRNeasy mini kit (Qiagen).

FIG. 4C is a plot showing nucleic acid (NA) levels in plasma and saliva of patients with oral squamous cell carcinoma, where healthy or tumor tissues (blood and saliva) were incubated in culture media for 24 hours before the supernatant was collected and the cfDNA was measured by PicoGreen.

FIG. 5A is a graph showing activation of HEK-Blue TLR Reporter cells (TLR 2) by patient tumor or healthy tissue supernatant.

FIG. 5B is a graph showing activation of HEK-Blue TLR Reporter cells (TLR 4) by patient tumor or healthy tissue supernatant.

FIG. 5C is a graph showing activation of HEK-Blue TLR Reporter cells (TLR 9) by patient tumor or healthy tissue supernatant.

FIG. 6A is a plot showing the generation of damage associated molecular pattern (DAMP) solution from HSC-3 cells.

FIG. 6B is a plot showing DNA binding efficiency of the NABNPs using calf thymus DNA in an EtBr competition assay.

FIG. 6C is a graph showing results of a Cck8 cytotoxicity assay of PAMAM-Chol NPs.

FIG. 6D, FIG. 6E and FIG. 6F are graphs showing TLR activation after treatment of HEK-BlueTM toll like receptor (TLR) expressing cells, with respective TLR agonists and DAMP solution at a polymer: agonist ratio.

FIG. 7 shows transwell migration invasion assay images of HSC-3 oral cancer cells treated with 1 μg/mL DAMP cfDNA and 50 μg/mL dendrimers.

FIG. 8 is a graph showing NFkB activation by HSC-3 freeze thaw DAMP solution in transfected HEK-293 cells, where TNF-a is used as a positive control and Dexamethosone as a NFkB inhibitor, and where DAMP solution is seen to activate NFkB in a concentration dependent manner.

FIG. 9 shows details of compound 1560, including the molecular formula and structure, molecular weight, logP, and pKa.

Similar reference characters denote corresponding features consistently throughout the attached drawings.

### PREPARING THE DRUG-LOADED NANOPARTICLES

### Manufacturing the Particles—Synthesis of PAMAM-Cholesterol(5) Polymer

PAMAM-G3 (104 mg, 15 μmol) in 5 mL methanol was mixed with cholesteryl chloroformate (34 mg, 75 μmol) in 5 mL dichloromethane. Then, N,N-Diisopropylethylamine (DIPEA, 39 mg, 300 μmol) was added. The mixture was stirred at 50° C. for 3 h, and dialyzed in ultrapure water for 72 h.

### Preferred Size of the Nanoparticles

The particles are preferably under 200 nm in size with a PDI (polydispersity index) under 0.3. Drug loading capacity is drug dependent, but testing with small hydrophobic drugs has demonstrated high loading capacity and efficiency.

### Process of Loading the Nanoparticles

400 μg Paclitaxel (PTX) was dissolved in 100 μL chloroform together with 1 mg PAMAM-Cholesterol(5) polymer. 2 mL water was added, and the mixture was sonicated for 2 minutes. An additional 5 mL water was added, and the excess solvent was removed by rotary evaporation. Unloaded drugs were eliminated by centrifugation of 3000 rpm for 30 min. The hydrodynamic diameter and zeta potential of the nanoparticles were measured using a Malvern Nano ZS90 Zetasizer.

Drug loading efficiency was determined by HPLC (Agilent 1260 infinity, USA). A reversed-phase HC-C18(2) column (4.6×150 mm, pore size 4 μm, Agilent, USA) was used. Nanoparticles of 1 mg were dissolved in 1 mL DCM (dichloromethane) under vigorous vortexing. The solvent was removed under vacuum, and the solid was redissolved in 10 mL of a 50/50 (v/v) mixture of acetonitrile and water for HPLC analysis. The mobile phase was a 38/31/31 (v/v/v) mixture of acetonitrile, methanol, and water with a flow rate of 1 mL min⁻¹. The absorbance at 227 nm was detected with a UV-Vis detector.

Note that while all drugs typically may be loaded using the same method, HPLC conditions for analysis will best be determined in a drug dependent manner

### BEST MODE(S)

The present subject matter relates to a composition for treatment in the form of PAMAM-Chol NPs, which may be used as a carrier for at least one drug. The at least one drug may be, as a non-limiting example, a chemotherapy drug, a protease-activated receptor 2 antagonist (e.g., I-343 or I-560), or a combination thereof. The at least one drug may also include at least one cancer drug, at least one corticosteroid, at least one anabolic steroid, at least one hormone (natural or synthetic), and combinations thereof. It should be understood that the PAMAM-Chol NPs may be used with any suitable type of drug or pharmaceutical composition, and are not limited to just the exemplary treatments discussed herein.

The present subject matter relates in part to leveraging cationic nanoparticles to mediate inflammation, while delivering various combinations of anti-nociceptive cargos to endosomal pain receptors in neurons and chemotherapeutics to cancer cells. This multi-focused therapy gives a novel approach to treating metastatic cancers, as it combats inflammation and damage associated molecular pattern (DAMP) induced metastasis while mediating pain in a non-opioid manner and delivering targeted chemotherapy. Following on our lab's original work with cationic polymers to mediate DAMP induced inflammation and the Bunnett Lab's original work showing that targeting endosomal receptor signaling provides an alternate and superior mechanism of managing pain, we have developed a scavenging multifunctional nanoparticle that can act as a superior form of therapy and delivery for cancer and other chronic diseases marked by pain and inflammation. Key features of these nanoparticles include: 1) ability to preferentially scavenge DAMPs; 2) inhibition of DAMP-mediated inflammation; 3) mediation of DAMP/toll like receptor (TLR) mediated metastasis; 4) controlled release delivery of drugs; and 5) inhibition of pronociceptive G protein-coupled receptors (GPCRs) in endosomes to magnify pain relief (e.g., neurokinin 1 receptor, calcitonin-like receptor, protease-activated receptor 2).

Cholesterol modified PAMAM-G3 nanoparticles have been developed to load cargos such as Taxol® and cisplatin (commonly used chemotherapeutics) as well as I-343 and I-560 (protease-activated receptor 2 antagonists). As used herein, “polyamidoamine-G3” refers to the third generation polyamidoamine dendrimer. The amide-rich surface of the nanoparticles (NPs) exhibits strong ability to adsorb cell-free DNA and RNA that can induce inflammation in tumor microenvironment. This nanoparticulate platform is able to mediate pro-inflammatory and pro-migratory TLR activation and provides a longer circulation time as well as a higher tumor targeting efficiency compared to free drug in mouse models. This technology will be developed further to package other pain receptor-specific drugs and therapeutics and may have broad applications in chronic pain and inflammation, e.g., inflammatory pain and cancer pain.

Non-limiting examples of the at least one cancer drug include coxorubicin, paclitaxel, camptothecin, docetaxel, pemetrexed, curcumin, gemcitabine, dabrafenib, dexamethasone, gefitinib, lenvatinib, methotrexate, thalidomide, vinblastine, vincristine, cyclophosphamide, ifosfamide, glyciphosphoramide, nimustine, carmustine, comustine, 5-fluorouracil, doxifluridine, mercaptopurine, cisplatin, and combinations thereof.

Non-limiting examples of the at least one corticosteroid include cortisone, prednisone, prednisolone, methylprednisolone, dexamethasone, betamethasone, hydrocortisone, and combinations thereof. Non-limiting examples of the at least one anabolic steroid include anadrol, oxandrin, dianabol, winstrol, deca-durabolin, equipoise, and combinations thereof.

Non-limiting examples of the at least one hormone include alclometasone, prednisone, dexamethasone, triamcinolone, cortisone, fludrocortisone, dihydrotachysterol, oxandrolone, oxabolone, testosterone, nandrolone, diethylstilbestrol, ethinyl estradiol, norethisterone, medroxyprogesterone acetate, hydroxyprogesterone caproate, estrogen, estradiol, estriol, estrone, cortisol, 11-deoxycortisol, aldosterone, corticosterone, 11-deoxycorti-costerone, aldosterone, progestin, pregnenolone, progesterone, 17α-hydroxy progesterone, 17α-hydroxy pregnenolone, dehydroepiandrosterone, androstenedoil, androstenedione, dihydrotestosterone, melatonin, thyroxine, and combinations thereof.

The nanoparticulate system may also be loaded with at least one drug intended specifically for pain relief. Non-limiting examples include NSAIDS (nonlimiting examples include Indomethacin, Sulindac, Etodolac, Tolmetin, Ketorolac, Oxaprozin, Fenoprofen, Flurbiprofen, Ibuprofen, Ketoprofen, Naproxen, Nambumetone, Meclofenamate, Diclofenac, Piroxicam, Meloxicam, Celecoxib, Rofecoxib, Valdecoxib, Aspirin, and combinations thereof), opioids (nonlimiting examples include Fentanyl, Alfentanil, Sufentanil, Remifentanil, Methadone, and combinations thereof), and local anesthetics (nonlimiting examples include Dibucaine, Bupivacaine, Lidocaine, Procaine, Mepivacaine, Rapivacaine, and combinations thereof). Suitable steroids, including without limitation the corticosteroids, anabolic steroids, and other steroids listed above, may also be useful for indirectly providing pain relief.

As a non-limiting example of dual treatment, the PAMAM-Chol NPs may be loaded with a cancer drug and a steroid. For example, steroids, such as dexamethasone, prednisolone, methylprednisolone, and/or hydrocortisone, are often given to patients to help reduce side effects of chemotherapy drugs, as well as reduce inflammation and help with appetite and energy levels. Thus, both a cancer drug (i.e., a chemotherapy drug) and a steroid can both be loaded on the PAMAM-Chol NPs. As a further alternative example, steroids or hormones could be loaded with one or more cancer drugs, and this combination could be loaded on the PAMAM-Chol NPs. It should, however, be understood that any suitable type of therapeutic agent or treatment may be loaded on the PAMAM-Chol NPs, and that the choice of drugs is not limited to the examples described above.

PAMAM-G3 was purchased from Sigma Aldrich® Inc. PAMAM-G3 was functionalized with cholesterol to form nanoparticles assembled by esterification. The cytotoxicity of the PAMAM polymers and nanoparticles was measured by MTT (3-(4, 5-dimethylthiazol-2-yl)-2,5-diphenyltetrazoliumbromide) assay. The agonists of toll-like receptors (TLR), including Pam3CSK4 (TLR2), LPS (TLR4), and CpG ODN (TLR9) were used to activate the TLR pathway-response HEK Blue™ reporter cells, respectively. Tissue and blood samples from oral cancer patients and DAMPs generated from oral cancer cell lines SCC-9 and HSC-3 were tested with the TLR reporters. Transwell-matrigel assay and cell wound healing (scratch-test) assay were used to determine the effects of NABNPs on inhibiting the invasion and migration of HSC-3 and SCC-9 cells induced by cfDNA, patient serum, and patient saliva.

The results show that PAMAM polymer and nanoparticles can effectively inhibit TLR activation from clinical samples. In a pilot study involving a small cohort of patients with oral tongue squamous cell carcinoma (OTSCC), we tested their tissue supernatants and saliva. The OTSCC tumor supernatants, on average, showed increased levels of cfDNA (1.43 μg/mL) compared to healthy tongue tissue (0.68 μg/mL). Saliva samples from patients with OTSCC also showed high levels of cfDNA at 4.75 μg/mL. When 100 ng/mL of tumor and normal tissue supernatants were used to activate TLR 2, 4, and 9 HEK reporter cells, we found that PAMAM polymer was able to mediate the overactivation of the TLR 4 and 9 pathways. These initial findings show that these nucleic-acid binding materials could effectively inhibit cfDNA-mediated cell inflammation through the TLR pathways. Further studies into migration and invasion preliminarily show that these polymers may be effective in slowing down the rate of tumor growth and invasion.

Thus, a nanomedicine, combining pain relief and efficient chemotherapeutic delivery with scavenging of pro-inflammatory cfDNA, may impact both cancer progression and its associated inflammation and/or pain. Our findings suggest that this may be a promising therapeutic strategy for combating oral cancer and potentially other inflammatory driven diseases. Table 1 below shows the characterization of the PAMAM-Chol nanoparticles (NPs).

TABLE 1
Characterization of PAMAM-Chol NPs

| | Terminal | Number | | | | Zeta |
|---|---|---|---|---|---|---|
| | Amino | of | % of | Mw/ | Diameter | Potential |
| Name | Groups | Grafting | Grafting | Da | (nm) | (mV) |
| PAMAM- | 32 | 0 | 0 | 6909 | — | — |
| G3 | | | | | | |
| PAMAM- | 32 | 2 | 6.25% | 7650 | 19.0 ± 4.7 | 44.3 ± 4.5 |
| Chol(2) | | | | | | |
| PAMAM- | 32 | 5 | 15.6% | 8762 | 20.1 ± 2.2 | 51.7 ± 0.9 |
| Chol(5) | | | | | | |

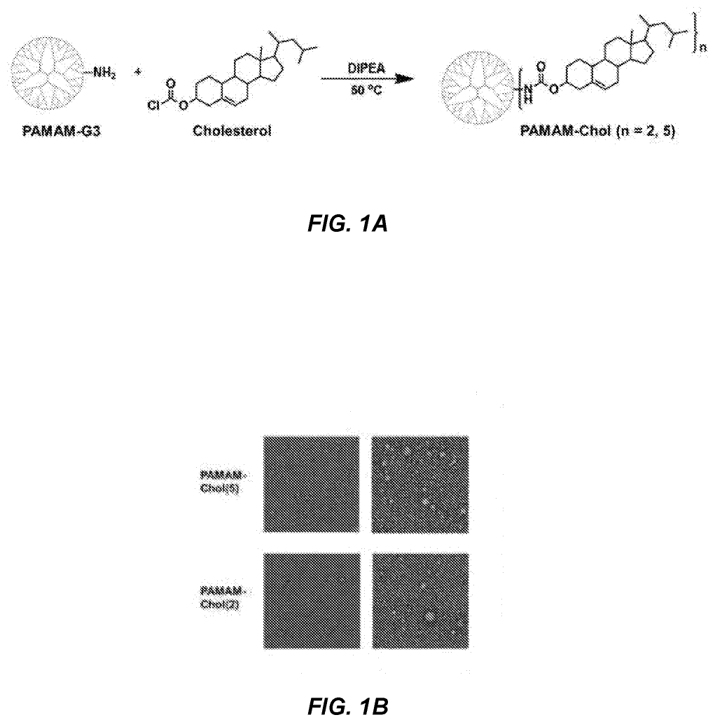

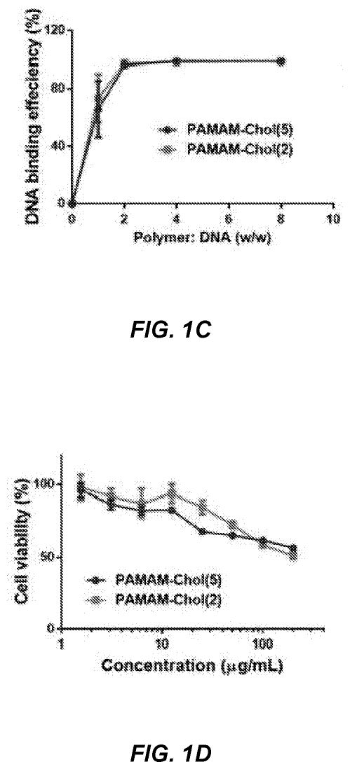

FIG. 1A illustrates the structure and formulation of polyamidoamine (PAMAM)-Chol. FIG. 1B is a set of transmission electron microscope (TEM) images of PAMAM-Chol NPs. FIG. 1C is a graph illustrating the binding efficiency of PAMAM-Chol NPs. FIG. 1D is a graph illustrating the results of a Cck8 cytotoxicity assay of PAMAM-Chol NPs.

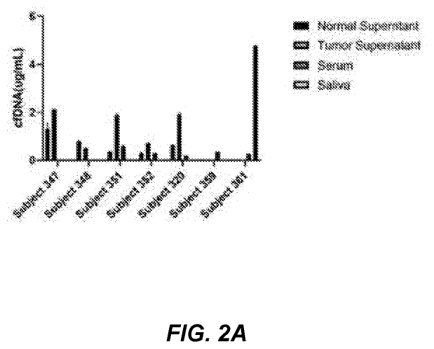

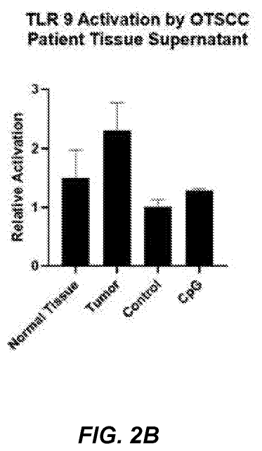

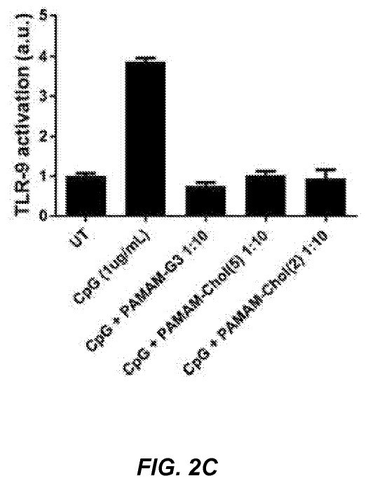

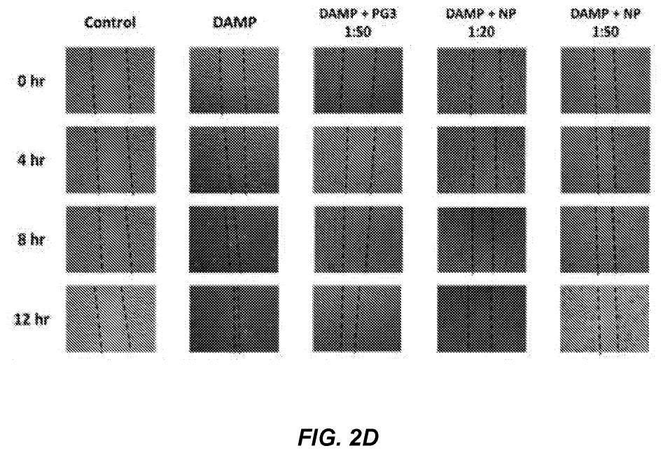

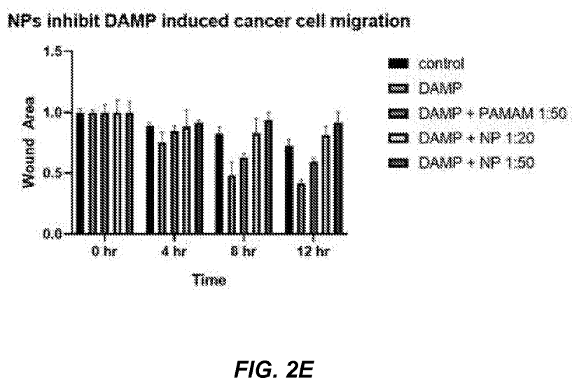

FIG. 2A is a graph showing cfDNA levels of human samples from patients with oral tongue squamous cell carcinoma (OTSCC). FIG. 2B is a graph showing activation of TLR 9 by OTSCC supernatants. FIG. 2C is a graph showing results of inhibition of TLR 9 activation by PAMAM-Chol NPs. FIG. 2D shows a set of wound-healing assay images, with the assay performed using confluent serum-starved HSC-3 cells either untreated or treated with 1 μg/mL cfDNA and 20 or 50 μg/mL dendrimers, illustrating that PAMAM-Chol NPs are able to successfully mediate cell migration. FIG. 2E is a graph showing a quantification of the wound healing assay of FIG. 2D, showing PAMAM-Chol NPs are successfully able to mediate damage associated molecular pattern (DAMP) induced cell migration, where the quantification is shown in the form of wound width relative to a control group, and produced using Image J software.

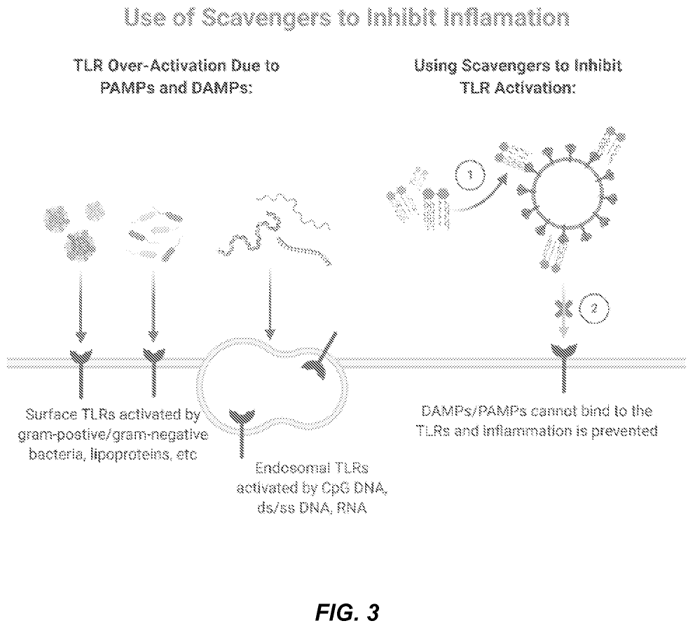

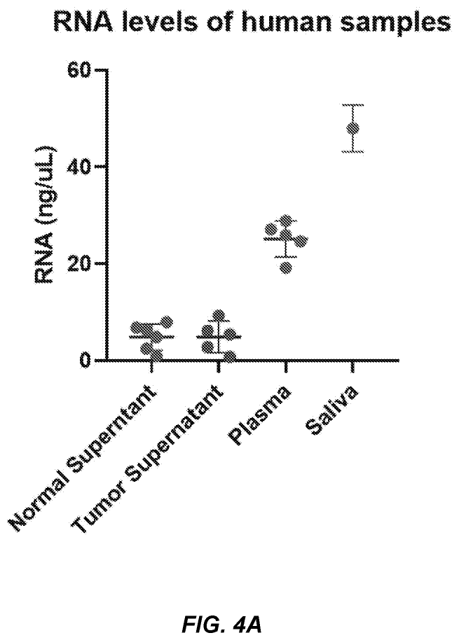

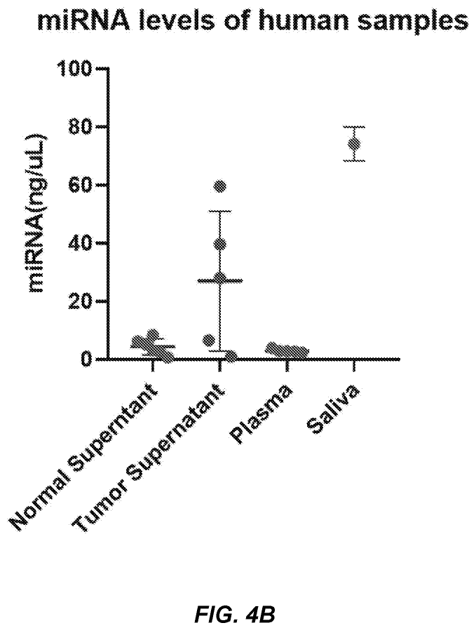

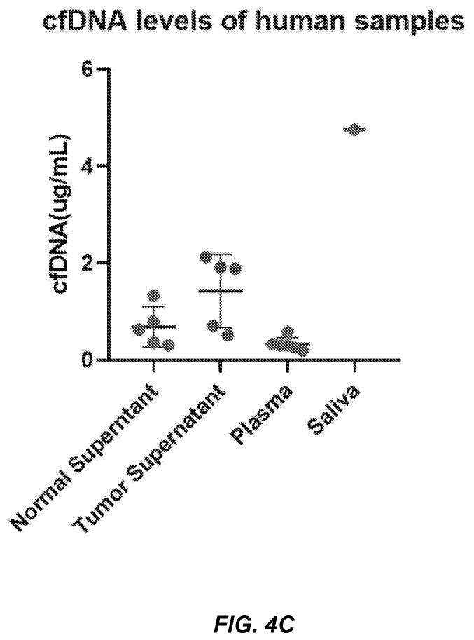

FIG. 3 diagrammatically illustrates the use of scavengers to inhibit inflammation. FIG. 4A is a plot showing nucleic acid (NA) levels in plasma and saliva of patients with oral squamous cell carcinoma, where healthy or tumor tissues (blood and saliva) were incubated in culture media for 24 hours before the supernatant was collected and the cfRNA was measured by Quanti-iT RNA (Thermo). FIG. 4B is a plot showing nucleic acid (NA) levels in plasma and saliva of patients with oral squamous cell carcinoma, where healthy or tumor tissues (blood and saliva) were incubated in culture media for 24 hours before the supernatant was collected and the miRNA was measured by a miRNeasy mini kit (Qiagen). FIG. 4C is a plot showing nucleic acid (NA) levels in plasma and saliva of patients with oral squamous cell carcinoma, where healthy or tumor tissues (blood and saliva) were incubated in culture media for 24 hours before the supernatant was collected and the cfDNA was measured by PicoGreen.

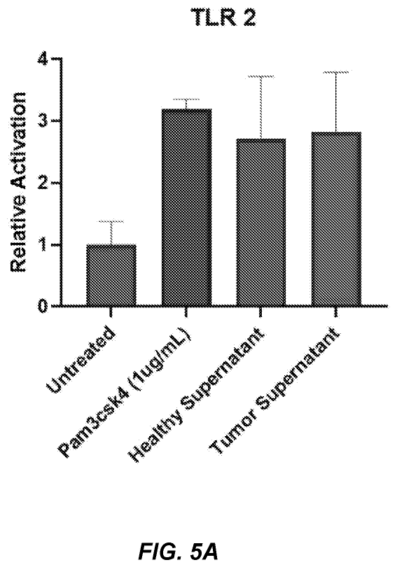

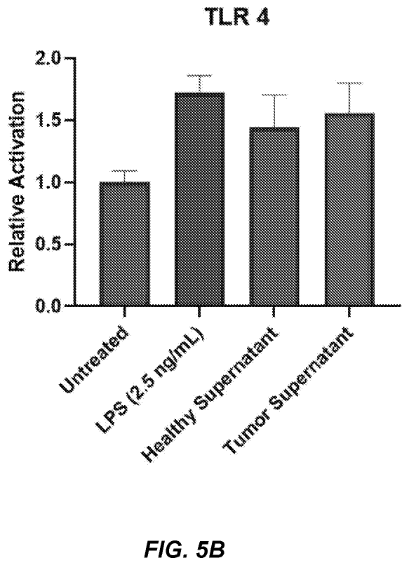

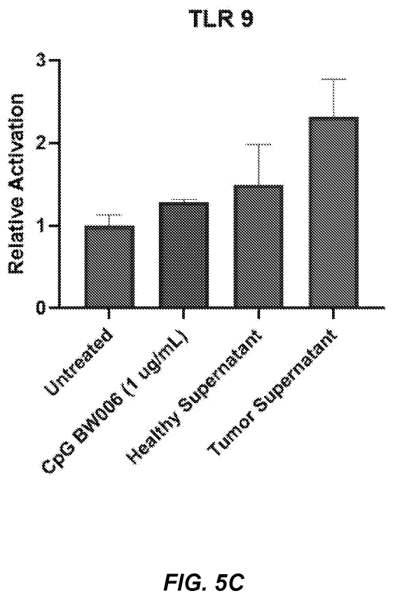

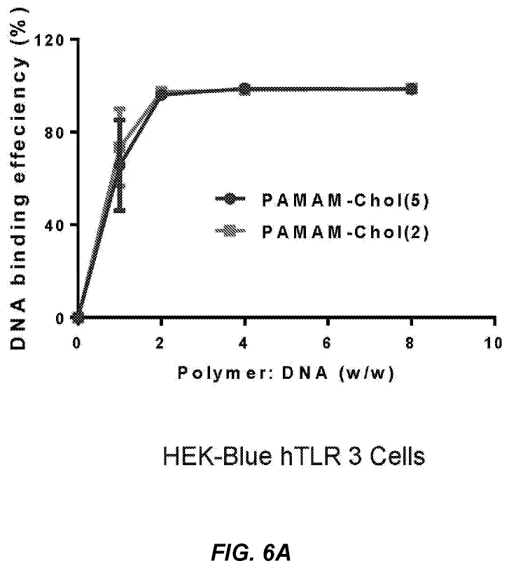

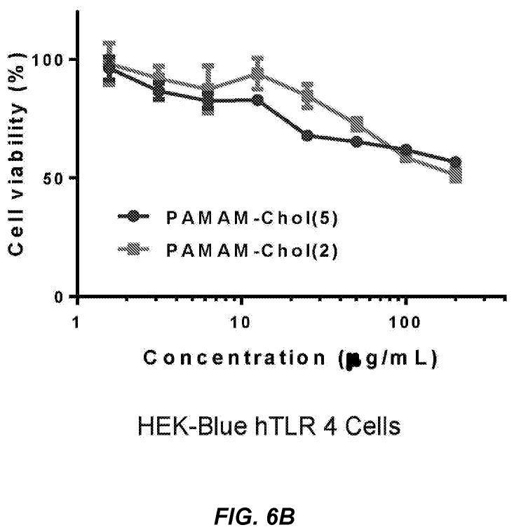

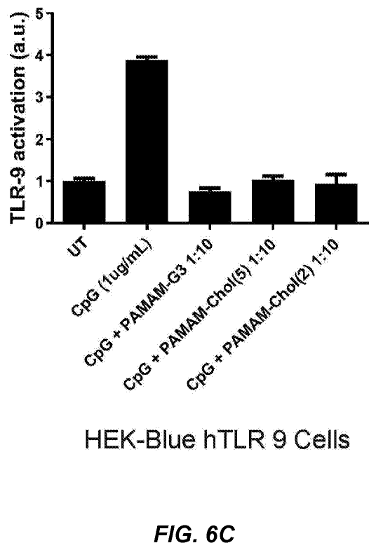

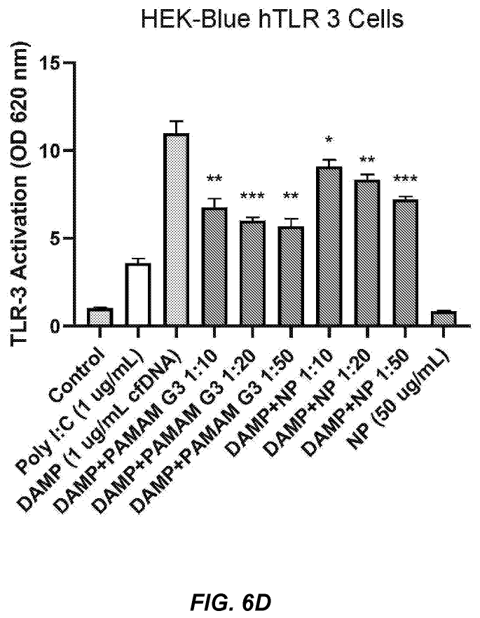

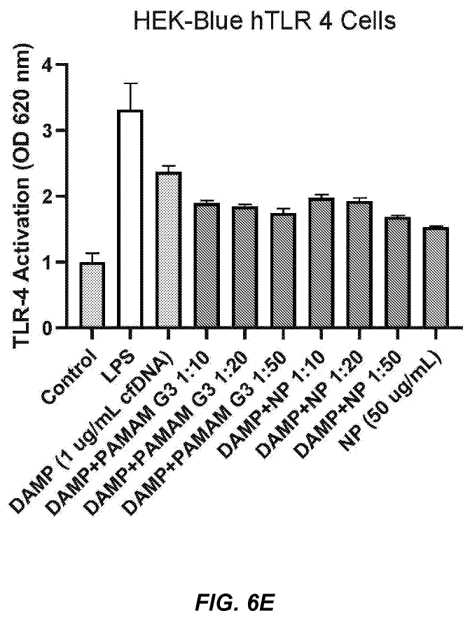

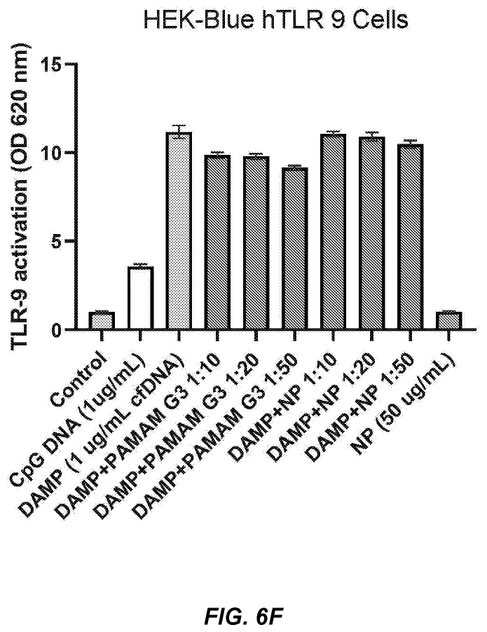

FIG. 5A is a graph showing activation of HEK-Blue TLR Reporter cells (TLR 2) by patient tumor or healthy tissue supernatant. FIG. 5B is a graph showing activation of HEK-Blue TLR Reporter cells (TLR 4) by patient tumor or healthy tissue supernatant. FIG. 5C is a graph showing activation of HEK-Blue TLR Reporter cells (TLR 9) by patient tumor or healthy tissue supernatant. FIG. 6A is a plot showing the generation of damage associated molecular pattern (DAMP) solution from HSC-3 cells. FIG. 6B is a plot showing DNA binding efficiency of the NABNPs using calf thymus DNA in an EtBr competition assay. FIG. 6C is a graph showing results of a Cck8 cytotoxicity assay of PAMAM-Chol NPs. FIG. 6D, FIG. 6E and FIG. 6F are graphs showing TLR activation after treatment of HEK-BlueTM toll like receptor (TLR) expressing cells, with respective TLR agonists and DAMP solution at a polymer: agonist ratio.

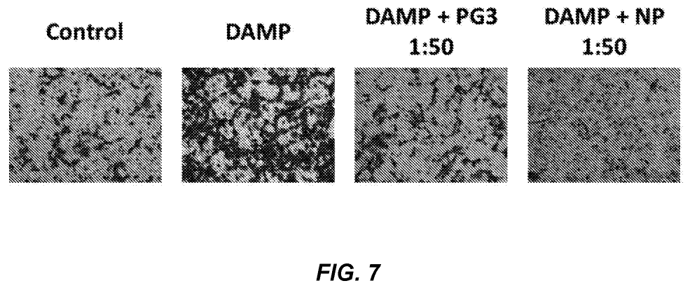

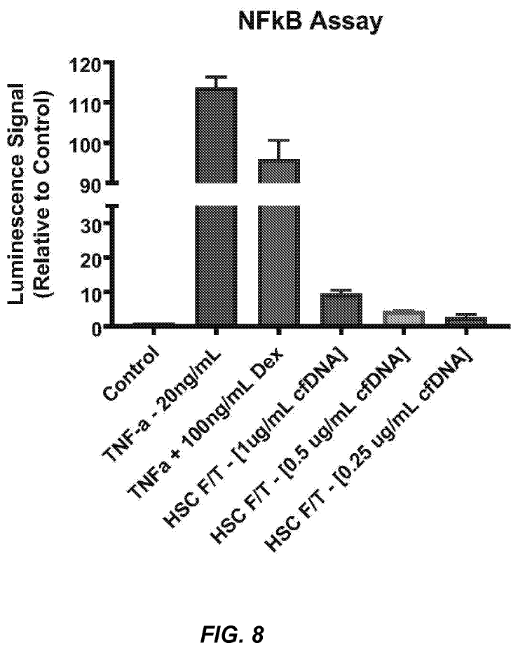

FIG. 7 shows transwell migration invasion assay images of HSC-3 oral cancer cells treated with 1 μg/mL DAMP cfDNA and 50 μg/mL dendrimers. FIG. 8 is a graph showing NFkB activation by HSC-3 freeze thaw DAMP solution in transfected HEK-293 cells, where TNF-a is used as a positive control and Dexamethosone as a NFkB inhibitor, and where DAMP solution is seen to activate NFkB in a concentration dependent manner.

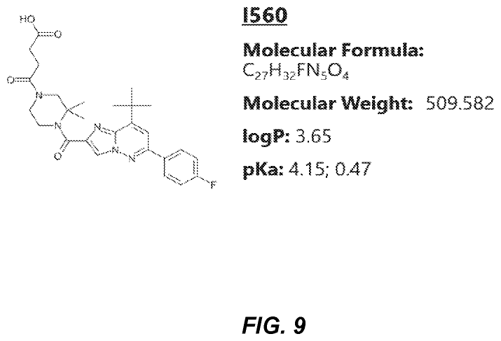

As discussed above, a non-limiting example of a chemotherapy drug that may be loaded on the PAMAM-Chol NPs is paclitaxel (sold under the brand name Taxol®). Table 2 below shows the loading and characterization data associated with paclitaxel-loaded PAMAM-Chol NPs (PC-PTX), or with compound 1560 (as described in FIG. 9), in comparison to PAMAM-Chol nanoparticles alone or conjugated with Cy5.

TABLE 2
Loading and Characterization data for Paclitaxel-Loaded PAMAM-Chol NPs

| | | | Zeta Potential | Loading | Loading |
|---|---|---|---|---|---|
| Nanoparticle | Size (nm) | PDI | (mV) | Capacity | efficiency |
| PAMAM- | 154.4 ± 4.685 | 0.203 ± 0.024 | 71.5 ± 1.99 | — | — |
| Chol NP | | | | | |
| PC-Cy5 | 167.9 ± 53.68 | 0.491 ± 0.124 | 58.3 ± 0.737 | — | — |
| PC-1560 | 171.4 ± 5.523 | 0.209 ± 0.014 | 58.6 ± 1.97 | 40% | 99% |
| PC-PTX | 169.9 ± 6.573 | 0.298 ± 0.029 | 53.3 ± 4.06 | 40% | 70% |

FIG. 9 depicts the chemical structure of compound 1560, as well as the molecular formula, molecular weight, logP, and pKa values.

### Example 1: NP Loaded with Compound 1560 (PC-1560)

400 μg of 1560 was dissolved in 200 μL chloroform together with 1 mg Pamam-Cholesterol polymer. 2 mL of water was added, and the mixture was sonicated for 2 minutes. An additional 4 mL water was added, and the excess solvent was removed by rotary evaporation. Unloaded drug was eliminated by dialysis against 9 mL of water +0.1% Tween-80 (v/v) for 1 hour. The hydrodynamic diameter and zeta potential of the nanoparticles were measured using a Malvern Nano ZS90 Zetasizer.

Drug loading efficiency was determined by HPLC (Agilent 1260 Infinity, USA). A reversed-phase HC-C18(2) column (4.6×150 mm, pore size 4 μm, Agilent, USA) was used. 1 mL of water from dialysis was dissolved in 1 mL DCM under vigorous vortexing. The solvent was removed under vacuum, and the solid was redissolved in acetonitrile for HPLC analysis. The mobile phase was a 60/40 (v/v) mixture of acetonitrile +0.1% trifluoroacetic acid (v/v) and water +0.1% trifluoroacetic acid (v/v) with a flow rate of 1 mL min⁻¹. The absorbance at 254 nm was detected with a UV-Vis detector. Drug release profiles were also determined by HPLC with the same condition.

### Example 2: NPs Conjugated with Cy5 (PC-Cy5)

Cy5-conjugated particles are fluorescent, and may be used, for example, in uptake and biodistribution studies. Pamam-Cholesterol nanoparticles were mixed with Cy5-NHS (cyanine5 NHS ester)) in a ratio of 50:1 by weight and shaken overnight at room temperature. Unloaded Cy5 was eliminated by dialysis against dialysis water for 72 hours, changing the water after 1, 2, 4, 24, and 48 hours. The resulting loaded nanoparticles were then stored at 4° C. until use.

It is to be understood that the nanoparticulate system for treating conditions including without limitation oral cancer and associated inflammation or pain is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A pharmaceutical treatment composition, comprising cholesterol-modified polyamidoamine-G3 nanoparticles loaded with at least one drug.

2. The pharmaceutical treatment composition as recited in claim 1, wherein the composition is formulated for treating cancer, and the at least one drug is selected from the group consisting of at least one cancer drug, at least one corticosteroid, at least one anabolic steroid, at least one hormone, and combinations thereof.

3. The cancer treatment composition as recited in claim 2, wherein the at least one drug comprises at least one cancer drug selected from the group consisting of coxorubicin, paclitaxel, camptothecin, docetaxel, pemetrexed, curcumin, gemcitabine, dabrafenib, dexamethasone, gefitinib, lenvatinib, methotrexate, thalidomide, vinblastine, vincristine, cyclophosphamide, ifosfamide, glyciphosphoramide, nimustine, carmustine, comustine, 5-fluorouracil, doxifluridine, mercaptopurine, cisplatin, and combinations thereof.

4. The cancer treatment composition as recited in claim 2, wherein the at least one drug comprises at least one corticosteroid selected from the group consisting of cortisone, prednisone, prednisolone, methylprednisolone, dexamethasone, betamethasone, hydrocortisone, and combinations thereof.

5. The cancer treatment composition as recited in claim 2, wherein the at least one drug comprises at least one anabolic steroid selected from the group consisting of anadrol, oxandrin, dianabol, winstrol, deca-durabolin, equipoise, and combinations thereof.

6. The cancer treatment composition as recited in claim 2, wherein the at least one drug comprises at least one hormone selected from the group consisting of alclometasone, prednisone, dexamethasone, triamcinolone, cortisone, fludrocortisone, dihydrotachysterol, oxandrolone, oxabolone, testosterone, nandrolone, diethylstilbestrol, ethinyl estradiol, norethisterone, medroxyprogesterone acetate, hydroxyprogesterone caproate, estrogen, estradiol, estriol, estrone, cortisol, 11-deoxycortisol, aldosterone, corticosterone, 11-deoxycorti-costerone, aldosterone, progestin, pregnenolone, progesterone, 17α-hydroxy progesterone, 17α-hydroxy pregnenolone, dehydroepiandrosterone, androstenedoil, androstenedione, dihydrotestosterone, melatonin, thyroxine, and combinations thereof.

7. A method of treating a disease or condition, comprising the step of administering to a patient in need thereof an effective amount of a treatment composition comprising cholesterol-modified polyamidoamine-G3 nanoparticles loaded with at least one drug.

8. The method of treating a disease or condition as recited in claim 7, wherein the method is a method of treating cancer.

9. The method of treating cancer as recited in claim 8, wherein the at least one drug is selected from the group consisting of at least one cancer drug, at least one corticosteroid, at least one anabolic steroid, at least one hormone, and combinations thereof.

10. The method of treating cancer as recited in claim 8, wherein the at least one drug comprises at least one cancer drug selected from the group consisting of coxorubicin, paclitaxel, camptothecin, docetaxel, pemetrexed, curcumin, gemcitabine, dabrafenib, dexamethasone, gefitinib, lenvatinib, methotrexate, thalidomide, vinblastine, vincristine, cyclophosphamide, ifosfamide, glyciphosphoramide, nimustine, carmustine, comustine, 5-fluorouracil, doxifluridine, mercaptopurine, cisplatin, and combinations thereof.

11. The method of treating cancer as recited in claim 8, wherein the at least one drug comprises at least one corticosteroid selected from the group consisting of cortisone, prednisone, prednisolone, methylprednisolone, dexamethasone, betamethasone, hydrocortisone, and combinations thereof.

12. The method of treating cancer as recited in claim 8, wherein the at least one drug comprises at least one anabolic steroid selected from the group consisting of anadrol, oxandrin, dianabol, winstrol, deca-durabolin, equipoise, and combinations thereof.

13. The method of treating cancer as recited in claim 8, wherein the at least one drug comprises at least one hormone selected from the group consisting of alclometasone, prednisone, dexamethasone, triamcinolone, cortisone, fludrocortisone, dihydrotachysterol, oxandrolone, oxabolone, testosterone, nandrolone, diethylstilbestrol, ethinyl estradiol, norethisterone, medroxyprogesterone acetate, hydroxyprogesterone caproate, estrogen, estradiol, estriol, estrone, cortisol, 11-deoxycortisol, aldosterone, corticosterone, 11-deoxycorti-costerone, aldosterone, progestin, pregnenolone, progesterone, 17α-hydroxy progesterone, 17α-hydroxy pregnenolone, dehydroepiandrosterone, androstenedoil, androstenedione, dihydrotestosterone, melatonin, thyroxine, and combinations thereof.

14. The pharmaceutical treatment composition as recited in claim 1, wherein the composition is formulated for treating or reducing inflammation.

15. The method of treating a disease or condition as recited in claim 7, wherein the method is a method of treating or reducing inflammation.
